(19) United States
(12) Patent Application Publication
(10) Pub. No.: US 2014/0363898 A1
(43) Pub. Date: Dec. 11, 2014

(54) METHOD FOR THE IDENTIFICATION OF BETA-SHEET AGGREGATED PROTEIN LIGANDS

(71) Applicant: Hoffmann-La Roche Inc., Nutley, NJ (US)

(72) Inventors: Edilio Borroni, Basel (CH); Christian Czech, Grenzach-Wyhlen (DE); Arnulf Dorn, Ruemmingen (DE); Luca Gobbi, Muttenz (CH); Valerie Goetschy-Meyer, Blotzheim (FR); Fiona Grueninger, Arlesheim (CH); Doris Roth, Allschwil (CH)

(73) Assignee: HOFFMANN-LA ROCHE INC., Nutley, NJ (US)

(21) Appl. No.: 14/137,399

(22) Filed: Dec. 20, 2013

Related U.S. Application Data
(63) Continuation of application No. PCT/EP12/61621, filed on Jun. 19, 2012.

(30) Foreign Application Priority Data
Jun. 22, 2011 (EP) 11170878.0

(51) Int. Cl. G01N 33/68

(52) U.S. Cl. CPC G01N 33/6896; G01N 2333/4709; G01N 2500/02
USPC 436/501

(57) ABSTRACT

The present invention provides an assay for the identification of beta-sheet aggregated protein ligands using Thiazine Red R.

### RELATED APPLICATIONS

This application is a continuation of International Application No. PCT/EP2012/061621, having an international filing date of Jun. 19, 2012, the entire contents of which are incorporated herein by reference, and which claims benefit under 35 U.S.C. §119 to European Patent Application No. 11170878.0, filed Jun. 22, 2011.

The present invention relates to a method for the identification of ligands which bind aggregated proteins forming beta sheets.

Tau protein aggregates in the brain of Alzheimer's patients correlate very well with the progression of neurodegeneration disease and could be useful as early imaging marker for a neurodegenerative process. There are other neurodegenerative diseases that show aggregation of tau. In combination with amyloid-beta imaging tauopathies could be differentiated from Alzheimer's disease. This will be a valuable tool for diagnosis and patients stratification. Tau imaging would be a useful biomarker for projects targeting tau pathology and neurodegeneration.

Therefore, there is a need for a method for the identification of PET (Positron Emission Tomography) imaging ligands for specific imaging of tau aggregates in Alzheimer's disease patients or in other tauopathies.

### BRIEF DESCRIPTION OF THE DRAWINGS

FIGS. 1 and 2 show saturation analyses of Thiazin red R for aggregated and monomeric proteins. There are two different binding sites for Thiazin-red on each of the aggregated proteins. Kd are determined for both binding sites (K_{d}1, K_{d}2) FIG. 1A is Tau; FIG. 1B is Abeta; and FIG. 2 is Alpha-synuclein.

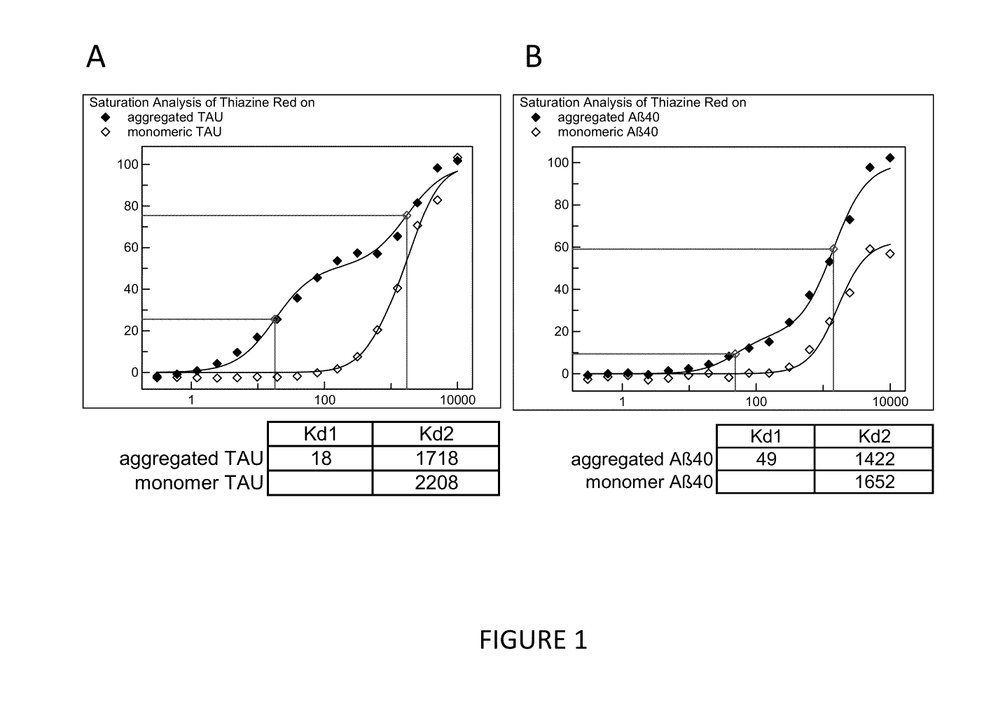

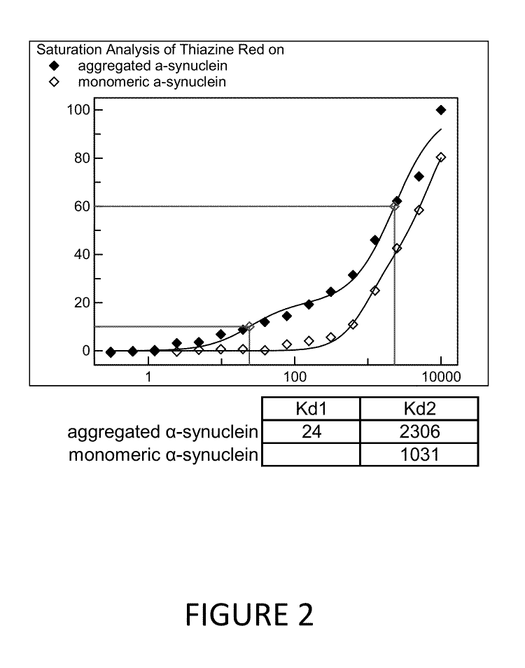

### DETAILED DESCRIPTION OF THE INVENTION

The method of the present invention is based on the finding that Thiazine Red R (CAS Nr. 2150-33-6) bound to beta-sheet aggregates shows a strong increase of fluorescence compared to Thiazine Red R bound to monomers forming the beta-sheet aggregates.

The term “beta-sheet aggregated protein” is used herein to refer to proteins which form aggregates having β-sheet structure. Tau protein, alpha-synuclein protein and Aβ peptide form aggregates having a β-sheet structure.

The term “protein” as used herein, refers to a polymer of amino acids, and not to a specific length. Thus, peptides, oligopeptides and protein fragments are included within the definition of polypeptide. The term “protein” is interchangeably used with the term “polypeptide”.

The term “tau protein” is used herein to refer to native sequence tau protein from any animal, e.g. mammalian, species, including humans, and tau variants (which are further defined below). The tau polypeptides may be isolated from a variety of sources, including human tissue types or prepared by recombinant and/or synthetic methods.

Natural or recombinantly produced tau protein can be used in this assay. A “recombinant protein” is a protein isolated, purified, or identified by virtue of expression in a heterologous cell, said cell having been transduced or transfected, either transiently or stably, with a recombinant expression vector engineered to drive expression of the protein in the host cell. Recombinant tau can be produced in procaryotic cells e.g. E. coli, in yeast e.g. S. pombe or in eukaryotic cells e.g. HEK 293, Sf9 insect cells. Preferably, Sf9 insect cells are used for high expression of recombinant tau. The tau protein used in the assay may be purified. The term “purified” as used herein refers to polypeptides, that are removed from their natural environment or from the source of recombinant production, isolated or separated, and are at least 60% and more preferably at least 80% free from other components, e.g. membranes and microsomes, with which they are naturally associated.

“Native sequence tau” refers to a polypeptide having the same amino acid sequence as a tau polypeptide occurring in nature regardless of its mode of preparation. A native sequence tau may be isolated from nature, or prepared by recombinant and/or synthetic methods. The term “native sequence tau” specifically encompasses naturally occurring truncated or secreted forms, naturally occurring variant forms (e.g. alternatively spliced forms), and naturally occurring allelic variants of tau. The identifier of the human tau polypeptide in the NCBI database is NP_{—}005901 (Seq. Id. No. 1).

The term “tau variant” refers to amino acid sequence variants of a native sequence tau, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence tau.

The term “Aβ peptide” is used herein to refer to native sequence Aβ peptide from any animal, e.g. mammalian, species, including humans, and Aβ peptide variants (which are further defined below). The Aβ peptide may be isolated from a variety of sources, including human tissue types or prepared by recombinant and/or synthetic methods.

Natural or recombinantly produced Aβ peptide can be used in this assay. A “recombinant protein” is a protein isolated, purified, or identified by virtue of expression in a heterologous cell, said cell having been transduced or transfected, either transiently or stably, with a recombinant expression vector engineered to drive expression of the protein in the host cell. Recombinant Aβ peptide can be produced in procaryotic cells e.g. E. coli, in yeast e.g. S. pombe or in eukaryotic cells e.g. HEK 293, Sf9 insect cells. The Aβ peptide used in the assay may be purified. The term “purified” as used herein refers to polypeptides, that are removed from their natural environment or from the source of recombinant production, isolated or separated, and are at least 60% and more preferably at least 80% free from other components, e.g. membranes and microsomes, with which they are naturally associated.

“Native sequence Aβ peptide” refers to a polypeptide having the same amino acid sequence as an Aβ peptide occurring in nature regardless of its mode of preparation. It is of note that “Aβ peptide” has several naturally occurring forms, whereby the human forms are referred to as Aβ39, Aβ40, Aβ41, Aβ42 and Aβ43. The most prominent form, Aβ42, has the amino acid sequence (starting from the N-terminus):

DAEFRHDSGYEVHHQKLVFFAEDVGSNKGAIIGLMVGGVVIA (Seq. Id. No. 3). In Aβ41, Aβ40, Aβ39, the C-terminal amino acids A, IA and VIA are missing, respectively. In the Aβ43-form an additional threonine residue is comprised at the C-terminus of the above depicted sequence (Seq. Id. No. 3). The preferred Aβ peptide used in the present assay is Aβ40 having the amino acid sequence given in Seq. Id. No. 4.

A native sequence Aβ peptide may be isolated from nature, or prepared by recombinant and/or synthetic methods. The term “native sequence Aβ peptide” specifically encompasses naturally occurring truncated or secreted forms, naturally occurring variant forms and naturally occurring allelic variants of Aβ peptide. The term “Aβ peptide variant” refers to amino acid sequence variants of a native sequence Aβ peptide, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence Aβ peptide.

The term “alpha-synuclein protein” is used herein to refer to native sequence alpha-synuclein protein from any animal, e.g. mammalian, species, including humans, and alpha-synuclein variants (which are further defined below). The alpha-synuclein polypeptides may be isolated from a variety of sources, including human tissue types or prepared by recombinant and/or synthetic methods.

Natural or recombinantly produced alpha-synuclein protein can be used in this assay. A “recombinant protein” is a protein isolated, purified, or identified by virtue of expression in a heterologous cell, said cell having been transduced or transfected, either transiently or stably, with a recombinant expression vector engineered to drive expression of the protein in the host cell. Recombinant alpha-synuclein can be produced in procaryotic cells e.g. E. coli, in yeast e.g. S. pombe or in eukaryotic cells e.g. HEK 293, Sf9 insect cells. Preferably, Sf9 insect cells are used for high expression of recombinant alpha-synuclein. The alpha-synuclein protein used in the assay may be purified. The term “purified” as used herein refers to polypeptides, that are removed from their natural environment or from the source of recombinant production, isolated or separated, and are at least 60% and more preferably at least 80% free from other components, e.g. membranes and microsomes, with which they are naturally associated.

“Native sequence alpha-synuclein” refers to a polypeptide having the same amino acid sequence as an alpha-synuclein polypeptide occurring in nature regardless of its mode of preparation. A native sequence alpha-synuclein may be isolated from nature, or prepared by recombinant and/or synthetic methods. The term “native sequence alpha-synuclein” specifically encompasses naturally occurring truncated or secreted forms, naturally occurring variant forms (e.g. alternatively spliced forms), and naturally occurring allelic variants of alpha-synuclein. The amino acid sequence of human alpha-synuclein polypeptide is given in Seq. Id. No. 2.

The term “alpha-synuclein variant” refers to amino acid sequence variants of a native sequence alpha-synuclein, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence alpha-synuclein.

The term “compound” is used herein in the context of a “test compound” or a “tracer candidate compound” described in connection with the assays of the present invention. As such, these compounds comprise organic or inorganic compounds, derived synthetically or from natural sources. The compounds include inorganic or organic compounds such as, but not limited to, polynucleotides, lipids or hormone analogs that are characterized by relatively low molecular weights.

Experimental Part

Recombinant human-microtubule associated protein Tau purified from E. coli is aggregated at a concentration of 5 μM with Arachidonic Acid (100 μM) in Tris 10 mM pH8, 24h at 37° C. Synthetic Aβ40 is aggregated with Arachidonic Acid (100 μM) in Tris 10 mM pH8, for three days at 37° C., under shaking at 150 rpm.

Human recombinant-Alpha-synuclein-purified from E. coli is aggregated with Arachidonic Acid (100 μM) in Tris 10 mM pH 8, for 5 days at 37° C., under shaking at 150 rpm.

A saturation analysis of Thiazin-red to the aggregated proteins is done to determine the affinity (Kd) of the Thiazin-red to the aggregated protein. Table 1 below shows the affinity constants of Thiazin-red for aggregated tau, Abeta and alpha-synuclein. The results show that there are two binding sites with different affinity on each aggregated protein for Thiazin-red.

TABLE 1

| | aggr. TAU | aggr. Aβ40 | aggr. α-syn |
|---|---|---|---|
| | IC50 (nM) | IC50 (nM) | IC50 (nM) |
| Evans Blue | 1 | 88.8 | 7.6 |
| Congo Red | 5.4 | 13.6 | 4.4 |
| Hondson 1d* | 8.8 | 13.2 | 3.6 |
| BSB | 18.4 | 78.4 | 36.3 |
| MeXO4 | 494.5 | 307.2 | 238.6 |
| Crystal Violet | 1545.7 | 1279.9 | 1715 |
| FDDNP | 1635.5 | 1467.4 | 1175.8 |
| IMPY | 2707.2 | 5671.2 | 5704.4 |
| PIB | 3255.3 | 5190 | 7015.1 |
| AZD2184 | 9801.9 | >10000 | >10000 |
| FENE | >10000 | >10000 | >10000 |
| BF-158 | >10000 | >10000 | >10000 |

Determination of Kd for different compounds on aggregated proteins using the Thiazine-red assay. Data represent average of different experiment .
*Compound 1d from Honson et al, Neurobiol Dis. 2007 Dec; 28(3):251-60.

Thiazin-red will be added at the concentration corresponding to the Kd to the respective aggregated protein binding site, to induce a fluorescent signal that can be inhibited by the addition of a displacer compound

To determine the affinity of a displacer compound to the Thiazin-red binding sites of the aggregated proteins, the compound is added at different concentrations in the assay ranging from 0.3 nM to 10000 nM.

In parallel, auto fluorescence of the compound is measured together with the aggregated proteins, but without Thiazin-red. As negative control, ligand and aggregated protein is used and as positive control, Thiazin-red, reference compound with known activity and aggregated protein is used.

Assay is performed in Perkin Elmer OptiPlate 384, black, 45 ul assay volume, assay buffer is DPBS no CaCl₂ no MgCl₂ (GIBCO N. 14020). Tested compounds are diluted in DMSO and 2 μl is added to the assay (5% DMSO final). Assay is started by the addition of the aggregated protein (competitive condition). Plates are shortly shacked (1 min with Sterico variomag teleshake) and incubated for 30 min at room temperature. Measurement are done with En:Vision (Perkin Elmer), at Excitation 531 nm/Emission 595 nm.

Table 1 shows the affinity constants of different compounds against aggregated tau, Abeta and alpha-synuclein against the high affinity binding site.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for the identification of a beta-sheet aggregated protein ligand comprising:
a) contacting a beta-sheet aggregated protein with Thiazine Red R and a compound,
b) measuring Thiazine Red R fluorescence in the mixture of step a), wherein a decreased Thiazine Red R fluorescence in the mixture of step a) compared to a blank is indicative for a beta-sheet aggregated protein ligand.

2. The method of claim 1, wherein the beta-sheet aggregated protein is selected from tau protein, Aβ peptide and alpha-synuclein protein.

3. The method of claim 1 or 2, wherein the beta-sheet aggregated protein is tau protein or Aβ40 peptide, preferably human tau protein or human Aβ40 peptide.

4. The method of claims 1 to 3, wherein the Thiazine Red R fluorescence is measured at Excitation 531 nm/Emission 595 nm.

5. The method of claims 1 to 4, wherein the beta-sheet aggregated protein is a recombinantly produced beta-sheet aggregated protein.

6. The method of claims 1 to 5, wherein the blank comprises the beta-sheet aggregated protein and Thiazine Red R.

7. The method of claims 1 to 6, wherein the compound is added to the mixture of step a) in increasing concentrations in order to determine the IC₅₀ of the test compound.

8. Use of Thiazine Red R for the identification of beta-sheet aggregated protein ligands.


SEQUENCE LISTING (4 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - protein, 42 aa, Homo sapiens [sequence omitted]

SEQ ID NO: 4 - protein, 40 aa, Homo sapiens
DAEFRHDSGYEVHHQKLVFFAEDVGSNKGAIIGLMVGGVV